(19) United States
(12) Patent Application Publication
(10) Pub. No.: US 2018/0153807 A1
(43) Pub. Date: Jun. 7, 2018

(54) COMPOSITIONS AND METHODS FOR TREATING MYCOBACTERIA INFECTIONS AND LUNG DISEASE

(71) Applicant: Matinas BioPharma Nanotechnologies, Inc., Bedminster, NJ (US)

(72) Inventor: Ruying Lu, New Providence, NJ (US)

(21) Appl. No.: 15/567,299

(22) PCT Filed: Apr. 22, 2016
(86) PCT No.: PCT/US16/28999

§ 371 Date: Oct. 17, 2017

Related U.S. Application Data
(60) Provisional application No. 62/151,218, filed on Apr. 22, 2015.

(51) Int. Cl. A61K 9/127; A61P 11/00; A61K 9/00; A61K 31/7036; A61K 47/02; A61K 47/24; A61P 31/04; A61P 31/06; A61K 47/28

(52) U.S. Cl. CPC A61K 9/1274; A61P 11/00; A61K 9/0053; A61K 31/7036; A61K 47/28; A61K 47/24; A61P 31/04; A61P 31/06; A61K 47/02

(57) ABSTRACT

Disclosed are methods of treating mycobacteria infections, such as nontuberculosis mycobacterial (NTM) lung disease and pulmonary tuberculosis, including refractory lung disease, by orally administering encochleated antibiotics, including aminoglycosides, such as amikacin. Orally administered antibiotic cochleates have significantly reduced toxicity as compared to intravenously administered antibiotics.

### CROSS REFERENCE TO RELATED APPLICATIONS

This application claims the benefit of, and relies on the filing date of, U.S. provisional patent application No. 62/151,218, filed 22 Apr. 2015, the entire disclosure of which is incorporated herein by reference.

### FIELD

This application relates generally to methods of administering encochleated antibiotics to treat mycobacteria infections.

### BACKGROUND

Organisms of the Mycobacterium genus are widely distributed in the environment, and can be found forming biofilms in water pipes and potable water tanks. Some Mycobacteria species are highly virulent and may spread from host to host by coughing and inhalation or direct contact, causing leprosy (M. leprae) or tuberculosis (M. tuberculosis). Other Mycobacteria species are significantly less virulent and are collectively referred to as Non-Tuberculous Mycobacteria (NTM). Although NTM are less virulent, under certain circumstances they may infect hosts with weakened immune systems or a particular physiology (such as bronchiectasis). Such infection of the host may occur by two different routes. One is the gastro-intestinal route, from where the bacteria can disseminate or/and cause lymph node infection. The other is the respiratory route, by which the bacterium causes infection in individuals with chronic pulmonary conditions (bronchiectasis, emphysema, cystic fibrosis, chronic obstructive pulmonary disease). The latter route of infection is common in individuals with underlying lung disease, and the lung infection is associated with the formation of biofilm (Carter G, et al, AAC 48:4907, 2004; Yamazaki Y, et al Cell Microbiol, 8: 808. 2006).

Amikacin (AMK) is an aminoglycoside antibiotic used most often to treat severe, hospital-acquired infections from multidrug-resistant gram-negative bacteria such as Pseudomonas aeruginosa, Acinetobacter and Enterobacter, as well as for the treatment of non-tuberculosis Mycobacterial (NTM) lung disease, typically for advanced lung disease and/or when first-line drugs fail to control the infection.

There is no oral form available as free amikacin is not absorbed orally, and amikacin, therefore must be given by intravenous (IV) or intramuscular (IM) routes. Liposomal amikacin for inhalation is currently in late stage clinical trials for the treatment of respiratory diseases, such as cystic fibrosis, Pseudomonasaeruginosa, non-tubercular mycobacterial infections and bronchiectasis.

Adverse side-effects of amikacin are similar to those of other aminoglycosides. Kidney damage and hearing loss are the most important effects. Because of this potential, blood levels of the drug and markers of kidney function (creatinine) are monitored.

Accordingly, the available routes for administering certain antibiotics, such as aminoglycosides, like amikacin, are limited and their administration must be closely monitored due to toxicity concerns. Providing an oral formulation for antibiotics like amikacin, and particularly one with a reduced toxicity profile, represents a major advancement in the treatment of mycobacterial infections, including NTM lung disease.

### SUMMARY

The experiments disclosed in this application demonstrate that aminoglycoside (e.g., amikacin) cochleates are effective at killing Mycobacteria avium in biofilm and in an animal model of nontuberculosis mycobacterial (NTM) lung disease. Thus the disclosure is directed, in part, to methods of treating a subject with a Mycobacterium avium infection, the method comprising orally administering to the subject a therapeutically effective amount of a formulation comprising a cochleate, wherein the cochleate comprises an aminoglycoside antibiotic.

One embodiment is directed to a method of treating a subject with a Mycobacteria lung disease, the method comprising orally administering to the subject a therapeutically effective amount of a formulation comprising a cochleate, wherein the cochleate comprises an antibiotic, such as an aminoglycoside antibiotic.

In one embodiment, the Mycobacteria lung disease is pulmonary tuberculosis. Typically, the pulmonary tuberculosis is caused by M. tuberculosis, M. bovis, M. africanum, M. microti and M. canetti.

In another embodiment, the Mycobacteria lung disease is nontuberculosis mycobacterial lung disease. In one embodiment, the NTM is selected from the group consisting of Mycobacterium avium, M. kanasasii, M. abscessus, M. xenopi, M. and M. intracellulare, and Mycobacterium avium complex.

In one embodiment, the method of treatment reduces bacterial load by at least 90%, 95%, or 98%. In one embodiment, the method of treatment reduces bacterial load by at least 90%, 95%, or 98% following at least 4 weeks of treatment.

In one embodiment, the subject has refractory lung disease following a standard course of therapy and before administration of the cochleate comprising the antibiotic.

In one embodiment, the subject has refractory lung disease following at least 6 months of a previous course of therapy and before administration of the cochleate comprising the antibiotic.

In one embodiment, the cochleate comprising the antibiotic is administered as monotherapy. In another embodiment, the cochleate comprising the antibiotic is administered as part of a multi-drug therapy.

In one embodiment, the multi-drug therapy comprises ethambutol and a macrolide, such as clarithromycin or azithromycin. In one embodiment, the multi-drug therapy further comprises rifamycin or rifampin.

In one embodiment, a no observed adverse effects level (NOAEL) of the aminoglycoside is greater than 100, 125, 150, 175, 200, 250, 300, 400, 500, 750, 1000, 1500, or 2000 mg/kg.

In one embodiment, the aminoglycoside antibiotic is selected from the group consisting of as amikacin, gentamycin, capreomycin, paromomycin, tobramycin, kanamycin, and neomycin. In one embodiment, the aminoglycoside antibiotic is amikacin.

In one embodiment, the antibiotic is administered at a dosage of between 5-20 mg/kg, alternatively 5-15 mg/kg, alternatively 5-10 mg/kg, alternatively 10-15 mg/kg, alternatively 10-20 mg/kg, alternatively 5-10 mg/kg, alternatively 5-25 mg/kg, or alternatively 1-30 mg/kg, In one embodiment, the antibiotic is administered at a dosage of about 400-1000 mg/day, alternatively 200-2000 mg/day, alternatively 100-4000 mg/day, alternatively 300-800 mg/day, alternatively 400-800 mg/day, alternatively 200-800 mg/day, alternatively 100-600 mg/day, alternatively 200-600 mg/day, alternatively 400-600 mg/day, alternatively 300-700 mg/day.

In one embodiment, the cochleate formulation further comprises sodium chloride. In one embodiment, the cochleate formulation contains 1 mM to 1M or 0.5M to 1M sodium chloride.

In one embodiment, the cochleate formulation further comprises bile salts. In one embodiment, the cochleate formulation contains 0.1 mM to 100 mM or 0.1 mM to 0.5 mM bile salts. In one embodiment, the bile salt is one or more of the following: cholate, chenodeoxycholate, taurocholate, glycocholate, taurochenodeoxycholate, glycochenodeoxycholate, deoxycholate, or lithocholate.

In one embodiment, the cochleate formulation is administered once per day, twice per day, three times per day, or four times per day. In another embodiment, the cochleate formulation is administered once per week, twice per week, three times per week, or four times per week. In one embodiment, the cochleate formulation is administered daily for at least 4 weeks. In another embodiment, the cochleate formulation is administered daily for at least 1 month, 2 months, 3 months, at least 4 months, or at least 6 months.

In one embodiment, the subject is a mammal. In one embodiment, the subject is a human.

In one embodiment, the cochleate comprises one or more negatively charged lipids, wherein the one or more negatively charged lipids comprise between 40% to 70% of the total lipid in the cochleate. In one embodiment, the one or more negatively charged lipids comprise between 30% to 70%, 40% to 60%, 45% to 65%, 45% to 60%, or 45% to 55% of the total lipid in the cochleate. In one embodiment, the one or more negatively charged lipids comprise phosphatidylserine. In one embodiment, the phosphatidylserine is soy phosphatidylserine. In another embodiment, the phosphatidylserine is egg or bovine derived phosphatidylserine.

In one embodiment, the cochleate further comprise one or more neutral or cationic lipid or sterols. In one embodiment, the one or more neutral or cationic lipid or sterols are selected from the group consisting of phosphatidylcholine and sphingomyelin.

In certain embodiments, the method is directed to a method of treating a subject with Mycobacterium avium complex (MAC) lung disease, the method comprising orally administering to the subject a therapeutically effective amount of a formulation comprising a cochleate, wherein the cochleate comprises an aminoglycoside antibiotic (e.g., amikacin).

Another aspect is directed to a method of treating a subject with nontuberculous mycobacterial (NTM) lung disease, the method comprising orally administering to the subject a therapeutically effective amount of a formulation comprising a cochleate, wherein the cochleate comprises an antibiotic, such as an aminoglycoside antibiotic (e.g., amikacin).

Another aspect is directed to a method of treating a subject with pulmonary tuberculosis, the method comprising orally administering to the subject a therapeutically effective amount of a formulation comprising a cochleate, wherein the cochleate comprises an antibiotic, such as an aminoglycoside antibiotic (e.g., amikacin).

Another aspect is directed to a method of treating a subject with disseminated nontuberculous mycobacterial (NTM) disease, the method comprising orally administering to the subject a therapeutically effective amount of a formulation comprising a cochleate, wherein the cochleate comprises an antibiotic, such as an aminoglycoside antibiotic (e.g., amikacin).

Another aspect is directed to a method treating a subject with a M. leprae or M. lepromatosis infection, the method comprising orally administering to the subject a therapeutically effective amount of a formulation comprising a cochleate, wherein the cochleate comprises an antibiotic, such as an aminoglycoside antibiotic (e.g., amikacin).

Yet another aspect is directed to a method of treating a subject with nontuberculous mycobacterial (NTM) disease, the method comprising orally administering to the subject a therapeutically effective amount of a formulation comprising a cochleate, wherein the cochleate comprises an antibiotic, such as an aminoglycoside antibiotic (e.g., amikacin) and wherein the method of treatment reduces bacterial load by at least 90%, 95%, or 98%. In one embodiment, the method reduces bacterial load by at least 90%, 95%, or 98% following at least 4 weeks of treatment. In certain embodiments, the NTM disease is NTM lung disease. In other embodiments, the NTM disease is disseminated NTM disease.

### BRIEF DESCRIPTION OF THE DRAWINGS

The accompanying drawings, which are incorporated in and constitute a part of this specification, illustrate certain embodiments, and together with the written description, serve to explain certain principles of the compositions and methods disclosed herein.

FIG. 1 shows the effective treatment of Mycobacterium avium complex (MAC) biofilm in culture using amikacin cochleates.

FIG. 2 shows the effective treatment of nontuberculosis Mycobacterium lung disease in mice treated daily for 4 weeks with amikacin cochleates.

FIG. 3 shows that the no adverse effect limit (NOAEL) of encochleated amikacin in rats is twice as high as the NOAEL of injected amikacin.

FIGS. 4A-B show the pharmacokinetics of encochleated amikacin in male (A) and female (B) rats. In humans, peak (Cmax) plasma levels should not exceed 35 mcg/mL and trough plasma levels should not exceed 10 mcg/mL, which levels are indicated by the upper horizontal lines in FIGS. 4A-B.

### DETAILED DESCRIPTION

Reference will now be made in detail to various exemplary embodiments, examples of which are illustrated in the accompanying drawings and discussed in the detailed description that follows. It is to be understood that the following detailed description is provided to give the reader a fuller understanding of certain embodiments, features, and details of aspects of the invention, and should not be interpreted as limiting the scope of the invention.

### 1. Mycobacteria

Mycobacteria are a family of small, rod-shaped bacilli that can be classified into 3 main groups for the purpose of diagnosis and treatment. The first is Mycobacterium tuberculosis complex which can cause pulmonary tuberculosis and includes M. tuberculosis, M. bovis, M. africanum, M. microti and M. canetti. The second group is M. leprae and M. lepromatosis, which cause Hansen's disease or leprosy. The third group is nontuberculous mycobacteria (NTM), which include all the other mycobacteria that can cause lung disease resembling tuberculosis, lymphadenitis, skin disease, or disseminated disease. NTM include, but are not limited to, M. avium Complex (MAC), M. avium, M. kansasii, M. abscessus, M. chelonae, M. fortuitum, M. genavense, M. gordonae, M. haemophilum, M. immunogenum, M. intracellulare, M. malmoense, M. marinum, M. mucogenicum, M. nonchromogenicum, M. scrofulaceum, M. simiae, M. smegmatis, M. szulgai, M. terrae, M. terrae complex, M. ulcerans, and M. xenopi. MAC includes at least two mycobacterial species, M. avium and M. intracellulare. These two species cannot be differentiated on the basis of traditional physical or biochemical tests, but there are nucleic acid probes that can be used to identify and differentiate between the two species.

There are two major forms of NTM lung disease: cavitary disease and nodular/bronchiectatic disease. Cavitary disease typically presents in males in their late 40s and early 50s who have a history of cigarette smoking and, often, excessive alcohol consumption. If left untreated, this form of NTM lung disease is generally progressive within 1-2 years and can result in extensive cavitary lung destruction and respiratory failure. The nodular/bronchiectatic disease typically presents with nodular and interstitial nodular infiltrates frequently involving the right middle lobe or lingula, and typically in postmenopausal, nonsmoking, white females. This form of the disease tends to progress more slowly than the cavitary form.

In addition to NTM lung disease, NTM may also cause disseminated disease. Disseminated disease due to NTM is among the most common and severe infections found in individuals suffering from advanced HIV infection. The majority (greater than 90%) of NTM disseminated disease is caused by MAC, with the overwhelming majority of these cases caused by M. avium. Disseminated disease due to NTM in individuals infected with HIV typically only occurs in patients who are severely immunocompromised and have very low CD4+ T cell counts. Disseminated disease due to NTM has also been detected in immunocompromised organ transplant (e.g., renal or cardiac) patients or chronic corticosteroid use.

Pulmonary tuberculosis is an acute or chronic infection caused by Mycobacteria tuberculosis or sometimes other Mycobacteria strains (M. bovis, M. africanum, M. microti and M. canetti) and is characterized by pulmonary infiltrates, formation of granulomas with caseation, fibrosis, and cavitation.

Leprosy is a chronic, systemic infection characterized by progressive cutaneous lesions and caused by M. leprae, an acid-fast bacillus that attacks cutaneous tissue and peripheral nerves, producing skin lesions, infections, and deformities. Leprosy can also be caused by M. lepromatosis. Leprosy occurs in three distinct forms. Lepromatus leprosy is the most serious type and causes damage to the upper respiratory tract, eyes, and testes, as well as nerves and skin. Tuberculoid leprosy affects peripheral nerves and occasionally the surrounding skin, especially on the face, arm, legs, and buttocks. Borderline (dimorphous) leprosy has characteristics of both lepramatous and tuberculoid leprosies. Skin lesions in this type of leprosy are diffuse and poorly defined.

### 2. Cochleates and Methods of Making the Same

Cochleates are anhydrous, stable, multi-layered lipid crystals which spontaneously form upon the interaction of negatively charged lipids, such as phosphatidylserine, and calcium (see, for example, U.S. Pat. Nos. 4,078,052; 5,643,574; 5,840,707; 5,994,318; 6,153,217; 6,592,894, as well as PCT Publ. Nos. WO 200404/091572; WO 2004/091578; WO 2005/110361, WO 2012/151517, and WO2014/022414, and U.S. Pat. Publ. 2010/0178325; each of which is incorporated fully herein by this reference). Cochleates have a unique multilayered structure consisting of a large, continuous, solid, lipid bilayer sheet rolled up in a spiral or as stacked sheets, with no internal aqueous space. This unique structure provides protection from degradation for associated “encochleated” molecules. Since the entire cochleate structure is a series of solid layers, components within the interior of the cochleate structure remain intact, even though the outer layers of the cochleate may be exposed to harsh environmental conditions or enzymes. Divalent cation concentrations in vivo in serum and mucosal secretions are such that the cochleate structure is maintained. Hence, the majority of cochleate-associated molecules are present in the inner layers of a solid, stable, impermeable structure. Once within the interior of a cell, however, the low calcium concentration results in the opening of the cochleate crystal and release of the molecule that had been formulated into cochleates. Accordingly, cochleate formulations remain intact in physiological fluids, including mucosal secretions, plasma and gastrointestinal fluid, thereby mediating the delivery of biologically active compounds by many routes of administration, including oral, mucosal and intravenous.

Cochleates can be made using known methods including, but not limited to, those described in U.S. Pat. Nos. 5,994,318 and 6,153,217, the entire disclosures of which are incorporated herein by reference. In one embodiment, the method generally includes combining a pharmacologically active agent (e.g., an antibiotic, such as an aminoglycoside) with a lipid (preferably a negatively charged phospholipid, such as phosphatidylserine) in the presence of a solvent, adding an aqueous solution to form liposomes, and precipitating with a multivalent cation to form a cochleate. In another embodiment, the method generally includes combining a pharmacologically active agent (e.g., an antibiotic, such as an aminoglycoside) with a liposome in the presence of a solvent such that the pharmacologically active agent (e.g., an antibiotic, such as an aminoglycoside) associates with the liposome, and precipitating with a multivalent cation to form a pharmacologically active agent-containing cochleate.

In a preferred embodiment, the multivalent cation is a divalent metal cation, such as calcium, zinc, magnesium, and barium. In a preferred embodiment, the divalent metal cation is calcium.

The step of introducing a pharmacologically active agent (e.g., an antibiotic, such as an aminoglycoside) to a liposome in the presence of a solvent can be achieved in a variety of ways. In one embodiment, the pharmacologically active agent (e.g., an antibiotic, such as an aminoglycoside) is introduced by introducing a solution of the solvent and the pharmacologically active agent (e.g., an antibiotic, such as an aminoglycoside) to the liposome. Preferably, the liposome is in a liposomal suspension, preferably, an aqueous liposomal suspension. In a preferred embodiment, the solution with the antibiotic is introduced to the liposome by dropwise addition of the solution. In other embodiments, the solution can be added by continuous flow or as a bolus. In addition the solution may be introduced to dried lipid, with water added before, after or with the solution.

In another embodiment, the pharmacologically active agent (e.g., an antibiotic, such as an aminoglycoside) is introduced to the liposome prior to or after the solvent. For example, the pharmacologically active agent (e.g., an antibiotic, such as an aminoglycoside) may be introduced to a liposomal suspension that includes the solvent. The mixture can then be agitated, mixed, vortexed or the like to facilitate association of the pharmacologically active agent (e.g., an antibiotic, such as an aminoglycoside) with the liposome. The pharmacologically active agent (e.g., an antibiotic, such as an aminoglycoside) introduced may be in a powder or a liquid form.

An antioxidant (e.g., Vitamin E) can also be used in making cochleates. The antioxidant can be introduced with the pharmacologically active agent (e.g., an antibiotic, such as an aminoglycoside) or with the liposome. Preferably, it is incorporated into the liposomal suspension or a solution of the pharmacologically active agent (e.g., an antibiotic, such as an aminoglycoside) and solvent.

The liposome may be prepared by any known method of preparing liposomes. Thus, the liposomes may be prepared for example by solvent injection, lipid hydration, reverse evaporation, freeze drying by repeated freezing and thawing. The liposomes may be multilamellar (MLV) or unilamellar (ULV), including small unilamellar vesicles (SUV). The concentration of lipid in these liposomal solutions can be from about 0.1 mg/ml to 500 mg/ml. Preferably, the concentration of lipid is from about 0.5 mg/ml to about 50 mg/ml, more preferably from about 1 mg/ml to about 25 mg/ml.

The liposomes may be large unilamellar vesicles (LUV), stable plurilamellar vesicles (SPLV) or oligolamellar vesicles (OLV) prepared, e.g., by detergent removal using dialysis, column chromatography, bio beads SM-2, by reverse phase evaporation (REV), or by formation of intermediate size unilamellar vesicles by high pressure extrusion. Methods in Biochemical Analysis, 33:337 (1988).

Any suitable solvent can be used in these methods. Solvents suitable for a given application can be readily identified by a person of skill in the art. Preferably, the solvent is an FDA acceptable solvent. The solvent can be an organic solvent or an inorganic solvent. In one embodiment, the solvent is a water miscible solvent. In another embodiment, the solvent is water or an aqueous buffer. Other suitable solvents include but are not limited to dimethylsulfoxide (DMSO), a methylpyrrolidone, N-methylpyrrolidone (NMP), acetonitrile, alcohols, e.g., ethanol (EtOH), dimethylformamide (DMF), tetrahydrofuran (THF), and combinations thereof. In general, the pharmacologically active agent (e.g., an antibiotic, such as an aminoglycoside) concentration within the solvent is between about 0.01 mg/ml and 200 mg/ml. Preferably, the pharmacologically active agent (e.g., an antibiotic, such as an aminoglycoside) concentration is between about 0.05 mg/ml and about 100 mg/ml, more preferably between about 0.1 mg/ml and 20 mg/ml.

The solvent can optionally be removed, e.g., before the formation of liposomes, at the liposome stage and/or after the cochleates are formed. Any known solvent removal method can be employed. For example, solvent may be removed from the liposomal suspension by tangential flow and/or filtration and/or dialysis, or from the cochleates by washing, filtration, centrifugation, and/or dialysis. The cochleates can be washed, e.g., with buffer or water, optimally with calcium or another cation.

A size-regulating agent may be introduced during the method of making the cochleate. A size-regulating agent, as used herein, refers to an agent that reduces the particle size of a cochleate. As used herein, the term “particle size” refers to the particle diameter, or in case the particles are not spherical, to the largest extension in one direction of the particle. The particle size of cochleates can be measured using conventional methods, such as a submicron particle size analyzer. In certain embodiments, the size regulating agent is a lipid-anchored polynucleotide, a lipid-anchored sugar (glycolipid), or a lipid-anchored polypeptide. In other embodiments the size regulating agent is a bile salt, such as oxycholate, cholate, chenodeoxycholate, taurocholate, glycocholate, taurochenodeoxycholate, glycochenodeoxycholate, deoxycholate, or lithocholate. Bile salts are bile acids compounded with a cation, usually sodium. Bile acids are steroid acids found predominantly in the bile of mammals and are commercially available.

In certain embodiments, the size-regulating agent is added to the lipid or liposomes before formation of the precipitated cochleate. For example, in one embodiment, the size-regulating agent is introduced into a liposomal suspension from which cochleates will subsequently be formed (e.g., by addition of cation or dialysis). Alternatively, the size-regulating agent may be introduced to a lipid solution, before of after addition of a pharmacologically active agent.

Any suitable lipid can be used to make the cochleate. In one embodiment, the lipid includes one or more negatively charged lipids. As used herein, the term “negatively charged lipid” includes lipids having a head group bearing a formal negative charge in aqueous solution at an acidic, basic or physiological pH, and also includes lipids having a zwitterionic head group.

The cochleates can also include non-negatively charged lipids (e.g., positive and/or neutral lipids). Preferably, the cochleates include a significant amount of negatively charged lipids. In certain embodiments, a majority of the lipid is negatively charged. In one embodiment, the lipid is a mixture of lipids, comprising at least 50% negatively charged lipid. In another embodiment, the lipid includes at least 75% negatively charged lipid. In other embodiments, the lipid includes at least 85%, 90%, 95% or 98% negatively charged lipid. In yet other embodiments, the negatively charged lipid comprises between 30%-70%, 35%-70%, 40%-70%, 45%-65%, 45%-70%, 40%-60%, 50%-60%, 45%-55%, 45%-65%, or 45%-50% of the total lipid in the cochleate.

The negatively charged lipid can include soy-based lipids, other-legume-based lipids, egg-based lipids, bovine-based lipids, or porcine-based lipids. Preferably, the lipid includes phospholipids, such as soy-based phospholipids. The negatively charged lipid can include phosphatidylserine (PS), dioleoylphosphatidylserine (DOPS), phosphatidic acid (PA), phosphatidylinositol (PI), and/or phosphatidyl glycerol (PG) and or a mixture of one or more of these lipids with other lipids. Additionally or alternatively, the lipid can include phosphatidylcholine (PC), phosphatidylethanolamine (PE), diphosphotidylglycerol (DPG), dioleoyl phosphatidic acid (DOPA), distearoyl phosphatidylserine (DSPS), dimyristoyl phosphatidylserine (DMPS), dipalmitoyl phosphatidylglycerol (DPPG) and the like. In one embodiment, the phosphatidylserine is soy phosphatidylserine. In another embodiment, the phosphatidylserine is egg or bovine derived phosphatidylserine.

### 3. Antibacterial Agents

The cochleates for use in the methods described herein are associated with or loaded with an antibacterial agent (also referred to herein as an antibiotic). By way of example, the anti-bacterial agent can include, but is not limited to, one or more of the following: a protein synthesis inhibitors; a 30S initiation inhibitor, such as aminoglycoside antibiotics (including streptomycin, dihydrostreptomycin, neomycin, framycetin, paromomycin, ribostamycin, kanamycin, amikacin, arbekacin, bekanamycin, dibekacin, tobramycin, spectinomycin, hygromycin B, paromomycin, capreomycin, gentamicin, netilmicin, sisomicin, isepamicin, verdamicin, and astromicin); a 30S tRNA binding antibiotic, such as tetracyclines, glycylcyclines, or fluorocyclines (including doxycycline, chlortetracycline, clomocycline, demeclocycline, lymecycline, meclocycline, metacycline, minocycline, oxytetracycline, penimepicycline, rolitetracycline, tetracycline, tigecycline, or eravacycline); a 50S initiation inhibitor, such as oxazolidinone antibiotics (including eperezolid, linezolid, posizolid, radezolid, ranbezolid, sutezolid, and tedizolid); a peptidyl transferase, such as amphenicols or pleuromutilins (including chloramphenicol, azidamfenicol, thiamphenicol, florfenicol, retapamulin, tiamulin, and valnemulin); a transpeptidation/translocation antibiotic, such as macrolides, ketolides, fluoroketolides, lincosamides or streptogramins (including azithromycin, clarithromycin, dirithromycin, erythromycin, flurithromycin, josamycin, midecamycin, miocamycin, oleandomycin, okitamycin, roxithromycin, spiramycin, troleandomycin, tylosin, telithromycin, cethromycin, solithromycin, fidaxomicin, carbomycin A, kitasamycin, clindamycin, lincomycin, pirlimycin, pristinamycin, quinupristin, dalfopristin, and virginiamycin); an elongation factor inhibitor, such as steroid antibacterials (including fusidic acid); a peptidoglycan synthesis/transpeptidases inhibitor, such as a penicillin (including natural penicillins penicillin G and penicillin V; β-lactamase-resistant penicillins methicillin, nafcillin, oxacillin, cloxacillin and dicloxacillin; aminopenicillins ampicillin, amoxicillin, pivampicillin, hetacillin, bacampicillin, metampicillin, talampicillin and epicillin; carboxypenicillins carbenicillin, and ticarcillin; ureidopenicillins mezlocillin and piperacillin) or a cehphalosporin (including cephacetrile, cefadroxyl, cephalexin, cephaloglycin, cephalonium, cephaloradine, cephalothin, cephapirin, Cefatrizine, Cefazaflur, Cefazedone, cephazolin, cephradine, cefroxadine, ceftezole, cefaclor, cefonicid, cefprozil, cefuroxime, cefuzonam, cefmetazole, cefotetan, cefoxitin, loracarbef, cefbuperazone, cefmetazole, cefminox, cefotetan, cefoxitin, cefotiam, cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefoperazone, ceftazidime, latamoxef, cefclidine, cefepime, cefluprenam, cefoselis, cefozopran, cefpirome, cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin); a penems or carbapenem (including faropenem, ertapenem, doripenem, imipenem, meropenem, biapenem, and panipenem); a monobactam (including aztreonam, tigemonam, carumonam, nocardicin A); a glycopeptide antibiotic (including vancomycin, oritavancin, telavancin, teicoplanin, dalbavancin, and ramoplanin); a beta-lactamase inhibitor (including clavulanate, sulbactam, tazobactam, and avibactam), an other antibiotic (including: fosfomycin, cycloserine, bacitracin, colistin, polymyxin B, daptomycin, lysozyme, gramicidin, isoniazid, or teixobactin).

In certain embodiments, the antibacterial agent is an aminoglycoside, including, but not limited to, streptomycin, dihydrostreptomycin, neomycin, framycetin, paromomycin, ribostamycin, kanamycin, amikacin, arbekacin, bekanamycin, dibekacin, tobramycin, spectinomycin, hygromycin B, paromomycin, capreomycin, gentamicin, netilmicin, sisomicin, isepamicin, verdamicin, and astromicin. In one embodiment, the aminoglycoside is amikacin.

### 4. Pharmaceutical Compositions

The cochleates described herein can be prepared as a pharmaceutical composition. Suitable preparation forms for the pharmaceutical compositions disclosed herein include, for example, tablets, capsules, soft capsules, granules, powders, suspensions, emulsions, microemulsions, nanoemulsions, unit dosage forms, rings, films, suppositories, solutions, creams, syrups, transdermal patches, ointments and gels.

The pharmaceutical compositions can include other pharmaceutically acceptable excipients, such as, a buffer (e.g., Tris-HCl, acetate, phosphate) of various pH and ionic strength; an additive such as albumin or gelatin to prevent absorption to surfaces; a protease inhibitor; a permeation enhancer; a solubilizing agent (e.g., glycerol, polyethylene glycerol); an anti-oxidant (e.g., ascorbic acid, sodium metabisulfite, butylated hydroxyanisole); a stabilizer (e.g., hydroxypropyl cellulose, hydroxypropylmethyl cellulose); a viscosity increasing agent (e.g., carbomer, colloidal silicon dioxide, ethyl cellulose, guar gum); a sweetener (e.g. aspartame, citric acid); a preservative (e.g., Thimerosal, benzyl alcohol, parabens); a flow-aid (e.g., colloidal silicon dioxide), a plasticizer (e.g., diethyl phthalate, triethyl citrate); an emulsifier (e.g., carbomer, hydroxypropyl cellulose, sodium lauryl sulfate); a polymer coating (e.g., poloxamers or poloxamines, hypromellose acetate succinate); a coating and film forming agent (e.g., ethyl cellulose, acrylates, polymethacrylates, hypromellose acetate succinate); an adjuvant; a pharmaceutically acceptable carrier for liquid formulations, such as an aqueous (water, alcoholic/aqueous solution, emulsion or suspension, including saline and buffered media) or non-aqueous (e.g., propylene glycol, polyethylene glycol, and injectable organic esters such as ethyl oleate) solution, suspension, emulsion or oil; and a parenteral vehicle (for subcutaneous, intravenous, intraarterial, or intramuscular injection), including but not limited to, sodium chloride solution, Ringer's dextrose, dextrose and sodium chloride, lactated Ringer's and fixed oils.

In certain embodiments, the pharmaceutical composition comprises a salt, such as NaCl or a bile salt, such as oxycholate, cholate, chenodeoxycholate, taurocholate, glycocholate, taurochenodeoxycholate, glycochenodeoxycholate, deoxycholate, or lithocholate. Bile salts are bile acids compounded with a cation, usually sodium. Bile acids are steroid acids found predominantly in the bile of mammals and are commercially available. In one embodiment, the bile salts comprise cholate. In another embodiment, the bile salts comprises deoxycholate. In yet another embodiment, the bile salts comprise cholate and deoxycholate. In another embodiment, the bile salts consist of cholate and deoxycholate.

In certain embodiments, the concentration of NaCl is 1 mM to 1M, 1 mM to 0.5M, 1 mM to 0.1M, 1 mM to 50 mM, 10 mM to 100 mM, 10 mM to 50 mM, 0.1M to 1M, 0.1M to 0.5M, or 0.5M to 1M. In certain embodiments, the concentration of the bile salts is 1 mM to 100 mM, 1 mM to 50 mM, 1 mM to 25 mM, 1 mM to 10 mM, 1 mM to 5 mM, 0.1 mM to 5 mM, 0.1 mM to 1 mM, or 0.1 mM to 0.5 mM bile salts.

These excipients are provided by way of example and it will be known to those of skill in the art that there will be other or different excipients that can provide the same chemical features as those listed herein.

### 5. Dosage and Administration

A pharmaceutical composition comprising a cochleate, as disclosed herein, is formulated to be compatible with its intended route of administration. Methods to accomplish the administration are known to those of ordinary skill in the art. This includes, for example, injections, by parenteral routes such as intravenous, intravascular, intraarterial, subcutaneous, intramuscular, intraperitoneal, intraventricular, intraepidural, or others as well as oral, nasal, ophthalmic, rectal, or topical. Typically, the cochleate is administered orally, for example, by administering a suspension, a tablet, a capsule, a softgel or other oral dosage form.

In certain embodiments, the antibiotic (e.g., an aminoglycoside, such as amikacin) is administered at a dosage of between 5-20, 5-10 mg/kg, 5-15, 10-15, 10-13 mg/kg, 10-20 mg/kg, 5-25 mg/kg, or 1-30 mg/kg. Alternatively, the antibiotic (e.g., an aminoglycoside, such as amikacin) can be administered at a fixed dosage of about 200-1000 mg/day, 400-1000 mg/day, 200-800 mg/day, 300-800 mg/day, 400-800 mg/day, 500-700 mg/day, 200-2000 mg/day, 100-4000 mg/day, 100-600 mg/day, 200-600 mg/day, 400-600 mg/day, or 300-700 mg/day, including, but not limited to about 400, 500, 600, 700, 800, 900, 1000, 1500, 2000, 2500, 3000, 3500, or 4000 mg.

Due to lower toxicity, the encochleated antibiotic may be administered more frequently or for a longer duration than an intravenous antibiotic. In certain embodiments, the encochleated antibiotic may be administered once per day, twice per day, three times per day, or four times per day. In another embodiment, the cochleate formulation is administered once per week, twice per week, three times per week, or four times per week. In one embodiment, the encochleated antibiotic may be administered 2-3 times weekly. In other embodiments, the cochleate formulation is administered daily for at least 1, 2, 3, 4, 5, 6, 7, 8, 9, 10, 11, or 12 weeks. In another embodiment, the cochleate formulation is administered daily for at least 3 months, at least 4 months, or at least 6 months.

### 6. Methods of Treatment

The cochleates as described herein can be used in a method of treating a subject with a Mycobacteria infection. One embodiment is directed to a method of treating a subject with a Mycobacterium avium infection, the method comprising orally administering to the subject a therapeutically effective amount of a formulation comprising a cochleate, wherein the cochleate comprises antibiotic, such as an aminoglycoside, including, for example, amikacin. In certain embodiments, the subject has lung disease. The methods can also be used to treat other mycobacterial infections.

One aspect is directed to a method of treating a subject with disseminated nontuberculous mycobacterial (NTM) disease, the method comprising orally administering to the subject a therapeutically effective amount of a formulation comprising a cochleate, wherein the cochleate comprises an antibiotic, such as an aminoglycoside antibiotic (e.g., amikacin). Typically, the subject with disseminated NTM disease is an immunocompromised patient, such as a patient infected with HIV.

Another aspect is directed to a method treating a subject with a M. leprae or M. lepromatosis infection, the method comprising orally administering to the subject a therapeutically effective amount of a formulation comprising a cochleate, wherein the cochleate comprises an antibiotic, such as an aminoglycoside antibiotic (e.g., amikacin).

Another aspect is directed to a method of treating a subject with Mycobacteria lung disease, the method comprising orally administering to the subject a therapeutically effective amount of a formulation comprising a cochleate, wherein the cochleate comprises an antibiotic, such as an aminoglycoside antibiotic (e.g., amikacin). In one embodiment, the Mycobacteria lung disease is pulmonary tuberculosis. In another embodiment, the Mycobacteria lung disease is nontuberculosis mycobacterial (NTM) lung disease.

Thus, one embodiment is directed to a method of treating a subject with pulmonary tuberculosis, the method comprising orally administering to the subject a therapeutically effective amount of a formulation comprising a cochleate, wherein the cochleate comprises an antibiotic, such as an aminoglycoside antibiotic (e.g., amikacin). Typically, pulmonary tuberculosis is caused by M. tuberculosis, M. bovis, M. africanum, M. microti or M. canetti.

Another embodiment is directed to a method of treating a subject with nontuberculosis mycobacterial (NTM) lung disease, the method comprising orally administering to the subject a therapeutically effective amount of a formulation comprising a cochleate, wherein the cochleate comprises an antibiotic, such as an aminoglycoside, including, for example, amikacin. In certain embodiments, the NTM is selected from the group consisting of Mycobacterium avium complex, M. kanasasii, M. abscessus, M. xenopi, M. avium, and M. intracellulare. Typically, the NTM comprises at least Mycobacterium avium complex.

In certain embodiments, the method of treatment reduces bacterial load by at least 90%, 91%, 92%, 93%, 94%, 95%, 96%, 97%, 98%, 99%, or 100%. In certain embodiments, the method reduces bacterial load by at least 95%. In certain embodiments, the method reduces bacterial load by at least 98%. In one embodiment, the method reduces bacterial load by at least 90%, 91%, 92%, 93%, 94%, 95%, 96%, 97%, 98%, 99%, or 100% following at least 4 weeks of treatment. In certain embodiments, the method reduces bacterial load by at least 95% following at least 4 weeks of treatment. In certain embodiments, the method reduces bacterial load by at least 98% following at least 4 weeks of treatment.

Yet another aspect is directed to a method of treating a subject with nontuberculous mycobacterial (NTM) disease, the method comprising orally administering to the subject a therapeutically effective amount of a formulation comprising a cochleate, wherein the cochleate comprises an antibiotic, such as an aminoglycoside antibiotic (e.g., amikacin) and wherein the method of treatment reduces bacterial load by at least 90%, 91%, 92%, 93%, 94%, 95%, 96%, 97%, 98%, 99%, or 100%. In certain embodiments, the method reduces bacterial load by at least 95%. In certain embodiments, the method reduces bacterial load by at least 98%. In one embodiment, the method reduces bacterial load by at least 90%, 91%, 92%, 93%, 94%, 95%, 96%, 97%, 98%, 99%, or 100% following at least 4 weeks of treatment. In certain embodiments, the method reduces bacterial load by at least 95% following at least 4 weeks of treatment. In certain embodiments, the method reduces bacterial load by at least 98% following at least 4 weeks of treatment. In certain embodiments, the NTM disease is NTM lung disease. In other embodiments, the NTM disease is disseminated NTM disease.

In certain embodiments, the subject has refractory lung disease following a previous course of therapy and before administration of the cochleate comprising the antibiotic. As used herein, “refractory lung disease” refers to lung disease that does not respond (as measured microbiologically, clinically or radiographically) to an appropriate therapy. In certain embodiments, the subject has refractory lung disease following at least 1, 2, 3, 4, 5, 6, 7, 8, 9, 10, 11, or 12 months of a previous course of therapy and before administration of the cochleate comprising the antibiotic. Typically, the subject has refractory lung disease following at least 6 months of a previous course of therapy and before administration of the cochleate comprising the antibiotic. The previous course of therapy can be any therapy used for treating a Mycobacteria infection or NTM lung disease. Refractory lung disease can also refer to lung disease where sputum is converted to acid-fast bacilli culture negative after 12 months of an appropriate therapy.

Typically, the first-line or standard course of therapy for NTM lung disease comprises a multi-drug therapy with ethambutol and a macrolide, such as clarithromycin or azithromycin. The multi-drug therapy can further comprise rifamycin or rifampin. For more advanced disease or refractory disease, intravenous streptomycin or amikacin may be added to the multi-drug regimen.

Typically, the first-line or standard course of therapy for the nodular/bronchiectactic form of the disease typically includes a macrolide (e.g., clarithromycin, 1000 mg three times weekly or azithromycin, 500-600 mg three times weekly), ethambutol (25 mg/kg three times weekly), and rifampin (600 mg three times weekly).

Typically, the first-line or standard course of therapy for the cavitary form of the disease typically includes a macrolide (e.g., clarithromycin, 500-1000 mg daily or azithromycin, 250-300 mg daily), ethambutol (15 mg/kg daily), rifampin (450-600 mg daily), and optionally intravenous streptomycin or amikacin, typically for the first 2-3 months of therapy (e.g., 25 mg/kg 2-3 times weekly).

Typically, the first-line or standard course of therapy for advanced or severe or previously treated disease typically includes a macrolide (e.g., clarithromycin, 500-1000 mg daily or azithromycin, 250-300 mg daily), ethambutol (15 mg/kg daily), rifabutin (150-300 mg daily) or rifampin (450-600 mg daily), and intravenous streptomycin or amikacin, typically for the first 2-3 months of therapy (e.g., 25 mg/kg 2-3 times weekly).

Typically, the first-line or standard course of therapy for pulmonary tuberculosis includes one or more of isoniazid, rifampin, rifabutin, rifapentine, pyrazinamide, and ethambutol, which can be supplemented with intravenous or intramuscular streptomycin, amikacin, kanamycin, and capreomycin.

In certain embodiments, the cochleate comprising the antibiotic (e.g., an aminoglycoside, such as amikacin) is administered as monotherapy. In other embodiments, the cochleate comprising the antibiotic (e.g., an aminoglycoside, such as amikacin) is administered as part of a multi-drug therapy, including for example a standard course of therapy for treating NTM lung disease or pulmonary tuberculosis, such as the standard courses of therapy described herein.

In certain embodiments, the subject has relapsed following a previous course of therapy and before administration of the cochleate comprising the antibiotic. As used herein, “relapse” refers to recurrence of lung disease. In certain embodiments, the subject relapses following at least 1, 2, 3, 4, 5, 6, 7, 8, 9, 10, 11, or 12 months of a previous course of therapy and before administration of the cochleate comprising the antibiotic. The previous course of therapy can be any therapy used for treating a Mycobacteria infection, NTM lung disease, NTM disseminated disease, pulmonary tuberculosis, or leprosy, including the standard courses of therapy described above and those known in the art.

The subject is a human or a non-human mammal, such as a dog, a cat, or a farm animal. Typically, the subject is a human.

### 7. Reduced Toxicity

Oral administration of encochleated aminoglycosides, such as amikacin, exhibit reduced toxicity as compared to parenteral administration of non-encochleated aminoglycosides. Patients treated with parenteral, non-encochleated aminoglycosides must be under close clinical observation because of the potential ototoxicity and nephrotoxicity associated with their use. The safety of parenteral administration of non-encochleated aminoglycosides for treatment periods longer than 14 days has not been established.

For parenteral administration of non-encochleated aminoglycosides, neurotoxicity, manifested as vestibular and permanent bilateral auditory ototoxicity, can occur in patients with pre-existing renal damage and in patients with normal renal function treated at higher doses and/or for periods longer than those recommended. The risk of aminoglycoside-induced ototoxicity is greater in patients with renal damage. High frequency deafness usually occurs first and can be detected only by audiometric testing. Vertigo may occur and may be evidence of vestibular injury. Other manifestations of neurotoxicity may include numbness, skin tingling, muscle twitching and convulsions. The risk of hearing loss due to aminoglycosides increases with the degree of exposure to either high peak or high trough serum concentrations. Patients developing cochlear damage may not have symptoms during therapy to warn them of developing eighth-nerve toxicity, and total or partial irreversible bilateral deafness may occur after the drug has been discontinued. Aminoglycoside-induced ototoxicity is usually irreversible. Aminoglycosides are also potentially nephrotoxic. The risk of nephrotoxicity is greater in patients with impaired renal function and in those who receive high doses or prolonged therapy. The no observed adverse effects level (NOAEL) of intravenous amikacin is 100 mg/kg.

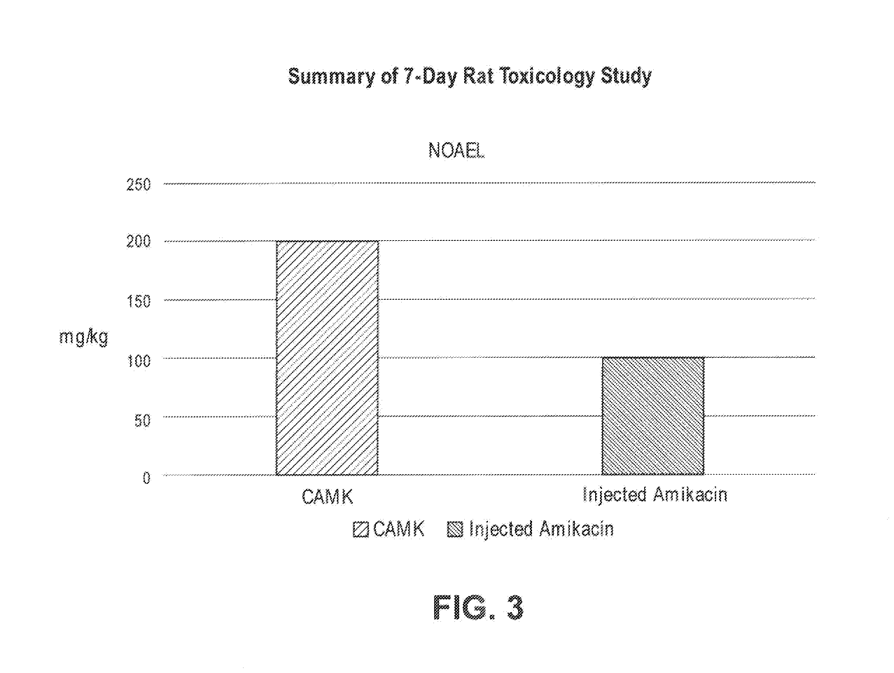

Administering aminoglycosides as part of a cochleate formulation reduces the toxicity associated with the aminoglycosides and permits the administration of higher doses of aminoglycosides. The NOAEL of a 7-day treatment regimen with encochleated amikacin in rats is 200 mg/kg or higher, two times the level of intravenous amikacin. FIG. 3. In certain embodiments, the NOAEL of the administered encochleated antibiotic (e.g., an aminoglycoside, such as amikacin) is greater than 100, 125, 150, 175, 200, 250, 300, 400, or 500 mg/kg.

Due to toxicity concerns, in humans, the peak (Cmax) plasma levels of amikacin should not exceed 35 μg/mL and the trough plasma levels should not exceed 10 μg/mL. In certain embodiments, the Cmax of the administered encochleated antibiotic (e.g., an aminoglycoside, such as amikacin) is between about 100-500 ng/mL (0.1-0.5 μg/mL) or about 100-400 ng/mL (0.1-0.4 μg/mL). The lower toxicity and lower Cmax values for encochleated antibiotics, such as amikacin, permits the antibiotics to be delivered orally at lower doses with improved efficacy and reduced toxicity. Alternatively, due to the lower toxicity, the encochleated antibiotic can be administered more frequently and/or at higher doses with less risk of adverse consequences.

### EXAMPLES

The examples provided below are simply for illustrative purposes. Those of skill in the art will be able to readily determine appropriate methods and equipment in order to produce suitable solid dispersion forms as described herein.

### Example 1: Cochleate Treatment of Mycobacteria Biofilm

System:

A549 alveolar epithelial cells were cultured in a transwell system. A549 cells became polarized after 6 days and integrity. Bacteria were seeded on the top (apical surface) of the cells. Seven days were allowed for biofilm formation.

Bacteria:

Mycobacterium avium complex (MAC) 104 (10⁵ bacteria) infection inoculum.

Treatment:

0.1 ml of the different treatments were delivered to the bottom well daily. The basolateral surface (bottom) of the cells were immersed in the tissue culture medium present in the bottom well. Three replicas per experimental group were tested in two different experiments.

Harvesting:

Biofilm and epithelial cells were lysed and diluted and plated onto 7H10.

| | CFU/bacteria, | |
|---|---|---|
| Experimental groups | 14 days | P value |
| No treatment | 7.4 ± 0.3 × 10⁶ | — |
| Empty cochleate | 6.9 ± 0.5 × 10⁶ | P > 0.05 |
| Free amikacin (100 μg/ml) | 3.2 ± 0.3 × 10⁵ | P = 0.02 |
| Free amikacin (20 μg/ml) | 9.1 ± 0.4 × 10⁵ | P < 0.05 |
| Cochleate-amikacin (0.66M NaCl) 100 μg/ml | 5.7 ± 0.4 × 10⁵ | P = 0.02 |
| Cochleate-amikacin (0.66M NaCl) 20 μg/ml | 1.8 ± 0.4 × 10⁶ | P < 0.05 |
| Cochleate-amikacin (2 mM bile salt) | 4.8 ± 0.3 × 10⁵ | P = 0.02 |
| 100 μg/ml | | |
| Cochleate-amikacin (2 mM bile salt) | 8.0 ± 0.5 × 10⁵ | P < 0.05 |
| 20 μg/ml | | |

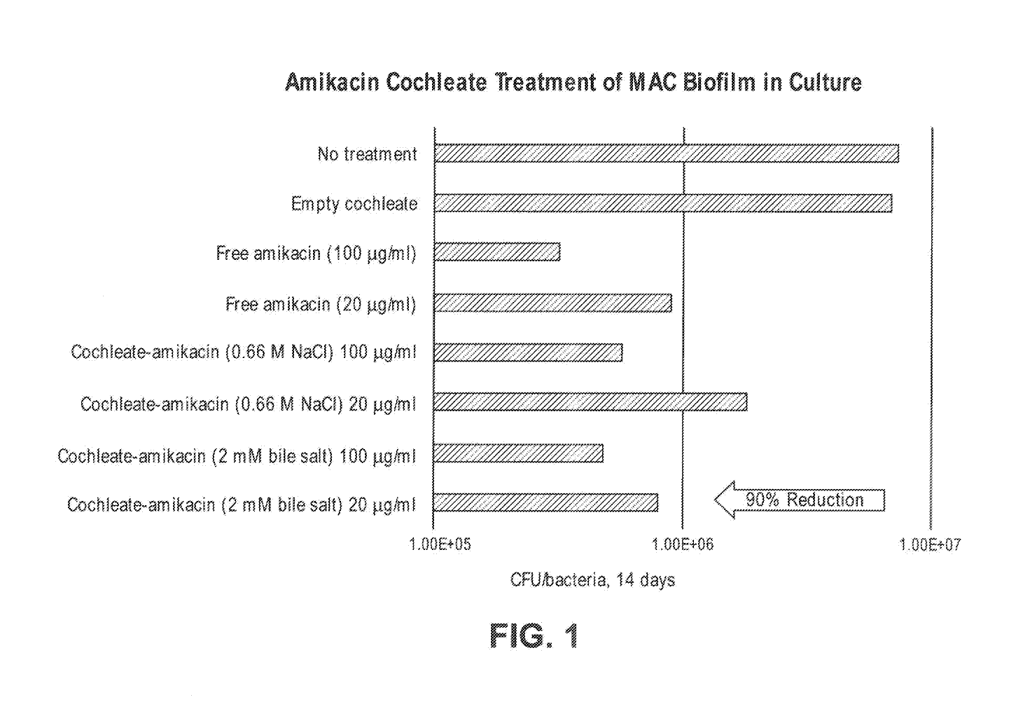

Conclusions: Biofilms of M. avium are encountered in lung infection. Although empty cochleate had no activity against M. avium in biofilm, both preparations of cochleates (sodium and bile salt) showed significant activity against M. avium in the model. FIG. 1. The anti-bacterial activity of the cochleates was similar to the activity of free amikacin (FIG. 1), suggesting that in absence of infected cells (small percent of the total infection), both preparations achieve comparable effect. Oral administration of encochleated amikacin shows anti-M. avium activity in biofilm.

### Example 2: Cochleate Treatment in Mice with Lung Disease

Experimental Model:

C57 BL/6 mice were infected with 8.3×10⁶ of Mycobacterium avium complex (MAC) intranasally and the infection was allowed to establish for 7 days. Then baseline bacterial load was determined in 10 mice and treatment protocols were initiated. Mice were treated daily for 4 weeks (orally or with intraperitonal injection of free amikacin). Mice were harvested and lung and spleens were removed, homogenized and plated to determine the bacterial load. Experimental groups had 12 mice each.

Bacteria:

8.3×10⁶MAC 104/HBSS

Results (bacterial load):

| | |
|---|---|
| Baseline | 5.5 ± 0.4 × 10⁵ |
| Empty cochleate | 4.3 ± 0.6 × 10⁵ |
| Free amikacin oral | 2.2 ± 0.5 × 10⁵⁽¹⁾ |
| Free amikacin IP 100 mg/Kg | 5.6 ± 0.4 × 10³⁽²⁾⁽³⁾ |
| Cochleate amikacin 100 mg/Kg/0.66M NaCl | 9.8 ± 0.6 × 10³⁽²⁾⁽³⁾⁽⁴⁾ |
| Cochleate amikacin 100 mg/Kg/2 mM bile | 9.1 ± 0.4 × 10³⁽²⁾⁽³⁾⁽⁴⁾ |
| salt | |
| Cochleate amikacin 100 mg/Kg/washed | 9.7 ± 0.3 × 10³⁽²⁾⁽³⁾⁽⁴⁾ |
| +Ca⁺⁺ | |

⁽¹⁾P > 0.05 compared to empty liposomes
⁽²⁾P > 0.05 compared to empty liposomes
⁽³⁾P < 0.05 compared to Baseline infection
⁽⁴⁾Approximately 75% of amikacin incorporated into cochleates

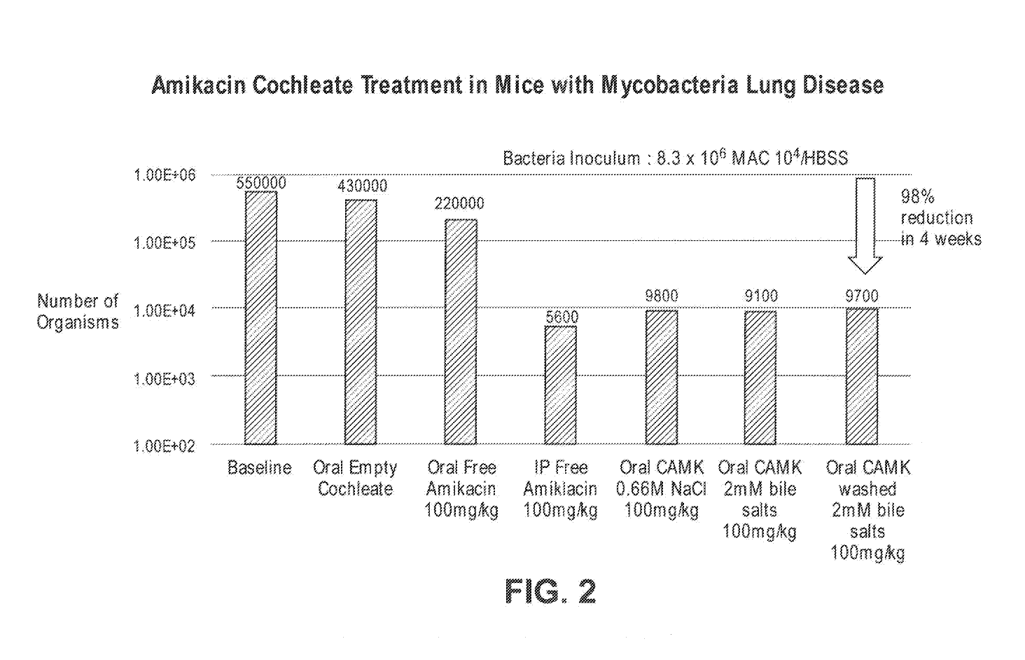

Free amikacin and cochleate amikacin preparations were effective for the treatment of lung infection by M. avium complex. FIG. 2. The cochleate amikacin 100 mg/kg 0.66 M NaCl, cochleate amikacin 100 mg/kg 2 mM bile salt (unwashed) and cochleate amikacin 100 mg/kg (washed plus Ca++) had very similar reduced CFU loads at 4 weeks (9.1 to 9.8×10³), which were all significantly two orders of magnitude lower than empty cochleates and baseline infection. The free 100 mg/kg IP AMK group had a CFU load remaining that was approximately half (5.6×10³) of that observed in the CAMK treatment groups. The effect observed was bactericidal for all the orally administered cochleate preparations and the free amikacin administered IP. Surprisingly, oral CAMK reduced the bacterial load by 98% following 4 weeks of daily treatment (FIG. 2), as compared to a reduction of only 76.3% (liver) and 86.7% (spleen) when oral CAMK was used to treat mice with disseminated M. avium infection. As noted above, the encochleation efficiency was about 75% for the CAMK treatment groups, which normalized the 100 mg/kg/day dose to 75 mg/kg/day.

Histopathology results suggested a similar nontoxicity among the CAMK and free IP AMK preparations. Overall, no toxicity was observed (including the histopathology of kidneys).

In conclusion, the in vivo anti-infectivity experiments against MAC in the C57 BL/6 mouse lung infection model suggest that both of the 2 mM bile salt preparations and the high salt 0.66 NaCl formulation had comparable efficacy as the free 100 mg/kg IP AMK formulation. Histopathology results suggested a similar nontoxicity among CAMK prepared with bile salts or high salt 0.66 M NaCl. Overall, no toxicity was observed (including the histopathology of kidneys).

### Example 3: In Vivo Toxicity Profile of Encochleated Amikacin

Amikacin (AMK) is a broad spectrum aminoglycoside commonly administered parenterally. Treatment with AMK is frequently associated with oto- and nephrotoxicity, therefore, careful monitoring of blood levels is required. To overcome issues associated with administration and toxicity of AMK, a cochleate formulation (CAMK) has been developed for oral delivery.

To assess the toxicity of CAMK, male and female Sprague Dawley rats (5/sex/group) were administered 50 mg/kg/day or 200 mg/kg/day oral dose of CAMK in 0.66 M NaCl, in 1 mM bile salts, or in 2 mM bile salts for 7 days. Free AMK was administered at 200 mg/kg/day oral and at 50 mg/kg/day intravenous (iv). Rats were euthanized on Day 8. Parameters that were evaluated include mortality/morbidity, clinical observations, body weights, plasma drug levels, toxicokinetic analysis, clinical pathology, necropsy observations, organ weights, and histopathology.

All animals survived to their scheduled sacrifice. There were no significant test article-related clinical observations during the treatment period except fecal stain and soft stool, mainly in males. There were no significant body weight changes in CAMK-treated groups.

No changes in hematology parameters were observed in any of the orally-treated groups that were attributed to treatment with amikacin formulations. In addition, no microscopic changes in bone marrow or spleen were noted.

Treatment with generic amikacin at 50 mg/kg iv yielded a reduction in RBC and HGB with an associated decrease in MCHC as well as increases in REW, RET, and REA in males and decreases in MCH, RDW, RET and REA in females. These changes may indicate compensatory erythropoiesis, though again, direct effects on the bone marrow were not observed microscopically.

Modest increases in cholesterol, triglycerides, total protein and globulin were observed sporadically in some of the groups treated with amikacin. These changes were generally small and not clearly dose-related. Changes in some of these levels may be associated with changes in food consumption, but the lack of differences in body weights between groups argues against this hypothesis. These changes may be related to modest changes in the liver, and significant decreases in liver weights were observed in several groups; however, these changes were likewise not clearly dose-related, and decreased liver size is inconsistent with the increases seen in these parameters. There were no microscopic changes seen in the liver that suggested any adverse effects of any of the amikacin formulations on the liver, suggesting that all of these changes are of minimal toxicologic significance.

Organ weight changes were mainly observed in CAMK-NaCl groups and in generic amikacin-treated groups. Decreases of 10-16% in heart, kidney and liver weights with corresponding decreases in organ-to-body or organ-to-brain weight ratios (9.5-15%) were seen in females treated with CAMK-NaCl at 200 mg/kg. With generic amikacin (AMK), increases in kidney- and spleen-to-body weight ratios (10-22%) were observed in males in the 50 mg/kg generic amikacin iv group, but not in females. These findings were not considered to be of any toxicological significance because of the lack of histopathological changes within these organs.

CAMK in three different vehicle formulations and vehicle control (empty cochleates) was well-tolerated by Sprague Dawley rats at 50 and 200 mg/kg for 7 days with no apparent effects on mortality/morbidity, clinical observations, body weights, food consumption, ophthalmology, or clinical pathology (hematology, clinical chemistry, and coagulation). Across dose groups, oral CAMK provided 100 fold lower plasma levels of AMK than the iv dose. There was a trend toward a lower exposure on Day 7 versus Day 1 in several dose groups. The amikacin prescribing information states that the peak plasma levels (Cₘₐₓ) of amikacin should not exceed 35 μg/mL and the trough plasma levels should not exceed 10 μg/mL. The Cₘₐₓ of all CAMK formulations did not exceed 0.5 μg/mL (500 ng/mL). FIG. 3.

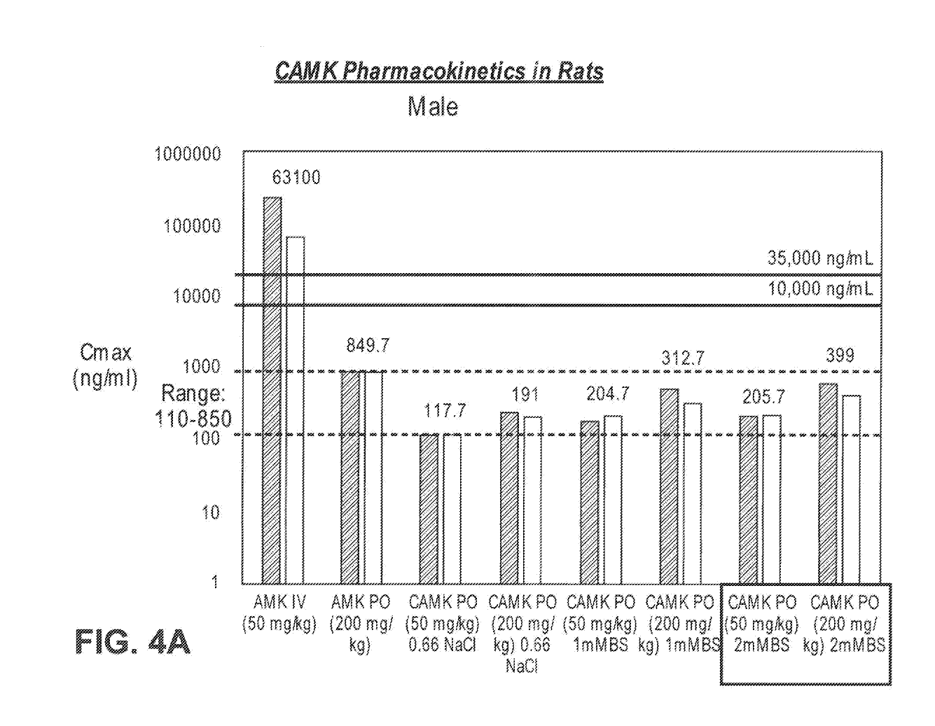

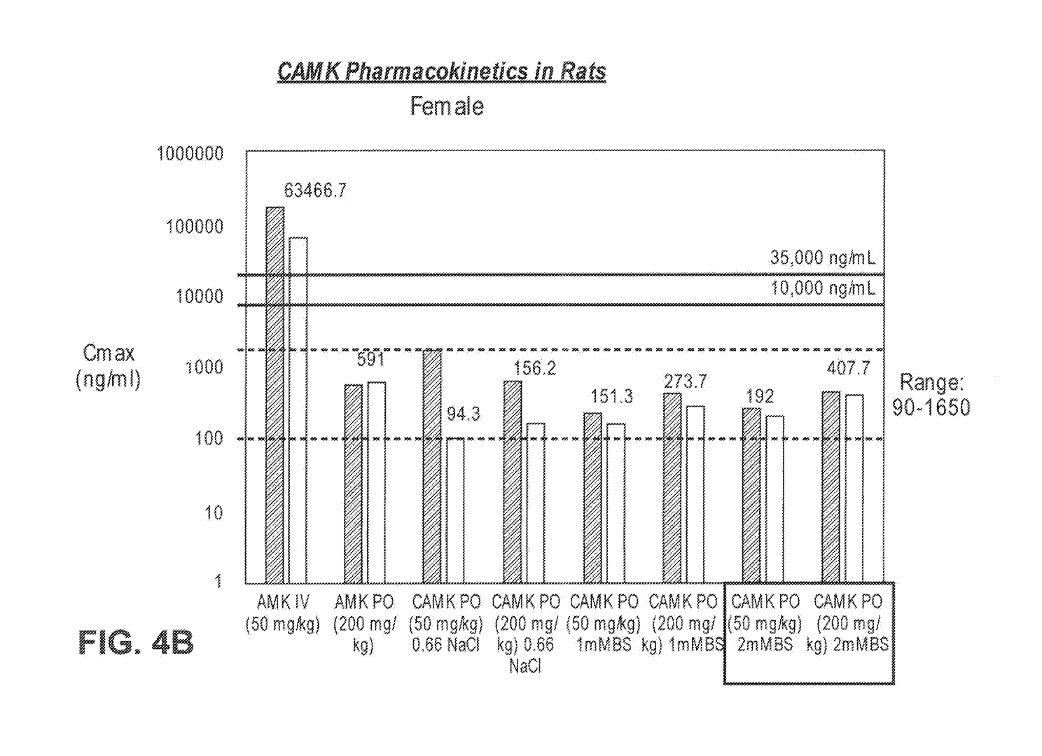

Based on changes in organ weights, the no adverse effect limit (NOAEL) is considered to be less than 50 mg/kg for CAMK in NaCl and 200 mg/kg for CAMK in 1 mM and 2 mM bile salt. FIG. 4. This compares favorably with the published NOAEL for injected amikacin in rats, which is 100 mg/kg. FIG. 4. The various changes seen in all treatment groups were of minimal toxicologic significance; therefore, the maximum tolerated dose (MTD) is considered to be greater than 200 mg/kg/day for all the dose formulations. This MTD is much higher than the currently recommended intravenous dose of 25 mg/kg three times weekly for patients with NTM lung disease.

Amikacin is typically administered intravenously (IV) or intramuscularly at 10-25 mg/kg/day for bacterial infections (approximately 300-500 mg as a single dose for a typical 70 kg human) to target plasma (or serum) peak and trough concentrations less than 35 μg/mL and 10 μg/mL, respectively. Following 7-day, repeat dose exposure of 200 mg/kg CAMK bile salts solution in rats, the systemic exposure of amikacin represented less than 2% of the systemic exposure following an IV 50 mg/kg dose. The NOAEL was considered to be 200 mg/kg for CAMK. Therefore, assuming similar oral bioavailability of CAMK in rats and humans, an 800 mg CAMK dose would be expected to produce peak concentrations in plasma less than the typical trough concentration observed following IV AMK, with trough CAMK concentrations at considerably lower levels. Using a conversion of 0.16 for body weight between rats and humans, the human equivalent dose (HED) of the NOAEL observed in rats would then translate to an HED of 32 mg/kg/day. For a typical 70 kg human, therefore, the NOAEL HED is 2240 mg/day. Applying a safety factor of approximately 10-fold, the minimum recommended starting dose is approximately 224 mg/day.

Those skilled in the art will recognize, or be able to ascertain using no more than routine experimentation, many equivalents to the specific embodiments of the invention described herein. Such equivalents are intended to be encompassed by the following claims.

1. A method of treating a subject with a Mycobacteria lung disease, the method comprising orally administering to the subject a therapeutically effective amount of a formulation comprising a cochleate, wherein the cochleate comprises an antibiotic.

2. The method of claim 1, wherein the Mycobacteria lung disease is pulmonary tuberculosis.

3. The method of claim 2, wherein the pulmonary tuberculosis is caused by M. tuberculosis, M. bovis, M. africanum, M. microti and M. canetti.

4. The method of claim 1, wherein the Mycobacteria lung disease is nontuberculosis mycobacterial (NTM) lung disease.

5. The method of claim 4, wherein the NTM is selected from the group consisting of Mycobacterium avium, M. kanasasii, M. abscessus, M. xenopi, M. avium, M. intracellulare, and Mycobacterium avium complex.

6. The method of claim 1, wherein bacterial load in the subject is reduced by at least 90%, 95%, or 98% following treatment.

7. The method of claim 1, wherein bacterial load in the subject is reduced by at least 90%, 95%, or 98% following at least 4 weeks of treatment.

8. The method of claim 1, wherein the subject has refractory lung disease following a standard course of therapy and before administration of the cochleate comprising the antibiotic.

9. The method of claim 8, wherein the subject has refractory lung disease following at least 6 months of a previous course of therapy and before administration of the cochleate comprising the antibiotic.

10. The method of claim 1, wherein the cochleate comprising the antibiotic is administered as monotherapy.

11. The method of claim 1, wherein the cochleate comprising the antibiotic is administered as part of a multi-drug therapy.

12. The method of claim 1, wherein the multi-drug therapy comprises ethambutol and a macrolide, such as clarithromycin or azithromycin.

13. The method of claim 12, wherein the multi-drug therapy further comprises rifamycin or rifampin.

14. The method of claim 1, wherein a no observed adverse effects level (NOAEL) of the aminoglycoside is greater than 100, 125, 150, 175, 200, 250, 300, 400, 500, 750, 1000, or 1500 mg/kg.

15. The method of claim 1, wherein the antibiotic is an aminoglycoside antibiotic.

16. The method of claim 15, wherein the aminoglycoside antibiotic is selected from the group consisting of amikacin, gentamycin, paromomycin, capreomycin, tobramycin, kanamycin, and neomycin.

17. The method of claim 15, wherein the aminoglycoside antibiotic is amikacin.

18. The method of claim 1, wherein the antibiotic is administered at a dosage of between 5-20 mg/kg, 5-15 mg/kg, 5-10 mg/kg, 10-15 mg/kg, 10-20 mg/kg, 5-10 mg/kg, 5-25 mg/kg, or 1-30 mg/kg.

19. The method of claim 1, wherein the antibiotic is administered at a dosage of about 400-1000 mg/day, 200-2000 mg/day, 100-4000 mg/day, 300-800 mg/day, 400-800 mg/day, 200-800 mg/day, 100-600 mg/day, 200-600 mg/day, 400-600 mg/day, or 300-700 mg/day.

20. The method of claim 1, wherein the cochleate formulation further comprises sodium chloride.

21. The method of claim 19, wherein the cochleate formulation contains 1 mM to 1M or 0.5M to 1M sodium chloride.

22. The method of claim 1, wherein the cochleate formulation further comprises bile salts.

23. The method of claim 22, wherein the cochleate formulation contains 0.1 mM to 100 mM or 0.1 mM to 0.5 mM bile salts.

24. The method of claim 1, wherein the bile salt is one or more of the following: cholate, chenodeoxycholate, taurocholate, glycocholate, taurochenodeoxycholate, glycochenodeoxycholate, deoxycholate, or lithocholate.

25. The method of claim 1, wherein the cochleate formulation is administered once per day, twice per day, three times per day, or four times per day.

26. The method of claim 1, wherein the cochleate formulation is administered daily for at least at least 1 month, 2 months, 3 months, at least 4 months, or at least 6 months.

27. The method of claim 1, wherein the subject is a mammal.

28. The method of claim 1, wherein the subject is a human.

29. The method of claim 1, wherein the cochleate comprises one or more negatively charged lipids, wherein the one or more negatively charged lipids comprise between 40% to 70% of the total lipid in the cochleate.

30. The method of claim 26, wherein the one or more negatively charged lipids comprise between 45% to 60% of the total lipid in the cochleate.

31. The method of claim 30, wherein the one or more negatively charged lipids comprise phosphatidylserine.

32. The method of claim 31, wherein the phosphatidylserine is soy phosphatidylserine.

33. The method of claim 29, wherein the cochleate further comprise one or more neutral or cationic lipid or sterols.

34. The method of claim 33, wherein the one or more neutral or cationic lipid or sterols are selected from the group consisting of phosphatidylcholine and sphingomyelin.

35. A method of treating a subject with nontuberculous mycobacterial (NTM) disease, the method comprising orally administering to the subject a therapeutically effective amount of a formulation comprising a cochleate, wherein the cochleate comprises an antibiotic, such as an aminoglycoside antibiotic (e.g., amikacin) and wherein the method of treatment reduces bacterial load in the subject by at least 90%, 95%, or 98%.

36. The method of claim 35, wherein the method reduces bacterial load by at least 90%, 95%, or 98% following at least 4 weeks of treatment.
